(19) United States
(12) Patent Application Publication
(10) Pub. No.: US 2011/0209713 A1
(43) Pub. Date: Sep. 1, 2011

(54) TOOTH COVER DEVICE FOR USE DURING ORAL SEX

(76) Inventor: Cheryl A. Lee, Newport Beach, CA (US)

(21) Appl. No.: 12/714,458

(22) Filed: Feb. 27, 2010

(51) Int. Cl. A61C 5/14; A61F 5/00

(52) U.S. Cl. 128/861; 600/38

(57) ABSTRACT

A tooth cover for covering a sexual partner's teeth when a male's erect penis is inserted into the mouth of the sexual partner during oral sex. The tooth cover has a flexible tooth cover body. There is a bottom surface that attaches to the sexual partners teeth and a top surface. The top surface provides a comfortable surface to receive the male's erect penis. A stick substance is connected to the bottom surface and is used to attach the bottom surface to the sexual partner's teeth. In a preferred embodiment, nodules are positioned on the top surface to provide a textured surface to better stimulate the male's erect penis. Also, in a preferred embodiment, the sticky substance is flavored to help mask any unpleasant taste of the male's erect penis.

The present invention relates to sexual aid devices, and in particular, to sexual aid devices for use during oral sex.

### BACKGROUND OF THE INVENTION

Human sexuality is a topic of high interest in society and, next to eating and drinking, it is arguably the most basic of human functions.

Although sexual intercourse is the most common form of sexual activity, oral sex is also very popular. Oral sex can be performed on a male or a female. When performed on a male, oral sex is defined as the insertion of a male's erect penis into the mouth of his sexual partner, as shown in FIG. 1.

The nerve endings of the erect penis are very sensitive so the male is able to receive tremendous pleasure during oral sex. Oral sex may serve as a prelude to sexual intercourse or it may be enjoyed entirely on its own culminating in the male achieving orgasm and ejaculating into the mouth of his sexual partner.

Despite the popularity of oral sex, if done improperly it can cause problems and discomfort for the male and his sexual partner. For example, as stated above nerve endings on the penis are very sensitive. Therefore if the erect penis contacts the sexual partner's teeth, the male can experience pain instead of pleasure. In an effort to ensure a pain free experience the sexual partner will therefore open her jaw widely so that the teeth do not contact the penis (FIG. 1). However, in order to provide adequate stimulation the sexual partner simultaneously will close her lips tight around the penis as it is thrust into her mouth. The simultaneous opening of the jaw and the closing of the lips can cause extreme discomfort and even injury to the sexual partner. This discomfort and the unpleasant taste of the male's penis and ejaculate are reasons often given by sexual partners for no longer wishing to engage in oral sex. Unfortunately, for a male who enjoys oral sex, it can cause a strain on their relationship if his sexual partner refuses to participate.

What is needed is a device for improving the ease and comfort level for performing oral sex.

### SUMMARY OF THE INVENTION

The present invention provides a tooth cover for covering a sexual partner's teeth when a male's erect penis is inserted into the mouth of the sexual partner during oral sex. The tooth cover has a flexible tooth cover body. There is a bottom surface that attaches to the sexual partners teeth and a top surface. The top surface provides a comfortable surface to receive the male's erect penis. A sticky/adhesive substance is connected to the bottom surface and is used to attach the bottom surface to the sexual partner's teeth. In a preferred embodiment, nodules are positioned on the top surface to provide a textured surface to better stimulate the male's erect penis. Also, in a preferred embodiment, the sticky substance is flavored to help mask any unpleasant taste of the male's erect penis.

### BRIEF DESCRIPTION OF THE DRAWINGS

FIG. 1 shows a prior art method of performing oral sex.

FIGS. 2-3 show a side view and front view of a method of utilizing a preferred embodiment of the present invention.

FIG. 4 shows a bottom view of a preferred embodiment of the present invention.

FIG. 5 shows a top view of a preferred embodiment of the present invention.

FIG. 6 shows a side view of a preferred embodiment of the present invention.

FIG. 7A shows a bottom view of the present invention with the tooth cover sticky substance covered by wax paper.

FIG. 7B shows a folded tooth cover.

FIGS. 8-10 show a method for donning a preferred embodiment of the present invention.

### DETAILED DESCRIPTION OF THE PREFERRED EMBODIMENTS

FIGS. 2 and 3 show the utilization of a preferred embodiment of the present invention. The sexual partner has placed tooth cover 3 over her upper and lower teeth. Tooth cover 3 includes nodules 4 for providing a stimulating texture to erect penis 5. Because the sexual partner's teeth are covered, the male no longer has to be concerned with direct contact with his sexual partner's teeth. Indeed, the pressure exerted through tooth cover 3 by the teeth only serve to increase the pleasure felt by the male. Moreover, the sexual partner is now able to relax her jaw and lips to avoid chances of injury or soreness and she is more able to thoroughly enjoy giving oral sex.

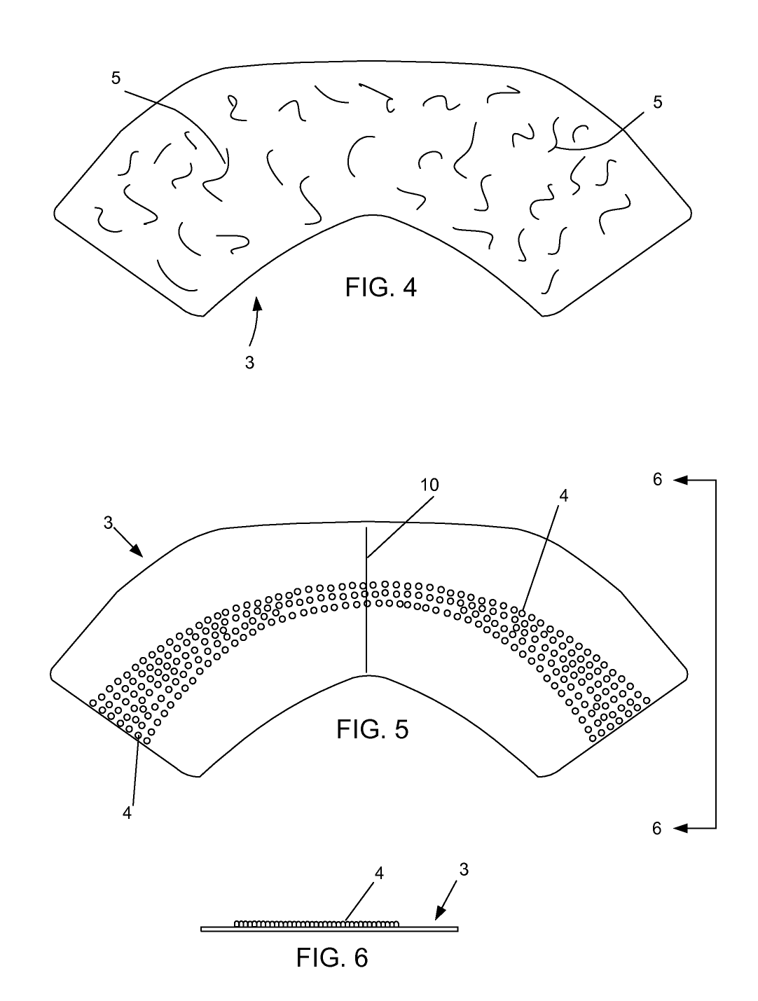

FIGS. 4-6 show a bottom view, top view and side view, respectively, of tooth cover 3. Tooth cover 3 is very thin and very flexible. It is preferably fabricated from polyurethane and is approximately 2 mm thick. Nodules 4 also extend an additional 2 mm out from the surface of tooth cover 3. Preferably, tooth cover 3 is made utilizing an injection molding process. The bottom of tooth cover 3 is coated with a sticky substance 5 to securely adhere tooth cover 3 to the sexual partner's teeth. In one preferred embodiment a denture adhesive is used as sticky substance 5. For example, POLIGRIP® can be used. POLIGRIP® is a federally registered trademark of the Stafford-Miller Corporation and refers in general to a denture adhesive. Also, as shown in FIG. 5, nodules 4 preferably extend so that they match the teeth line of the sexual partner.

### Preferred Method for Covering Teeth with the Tooth

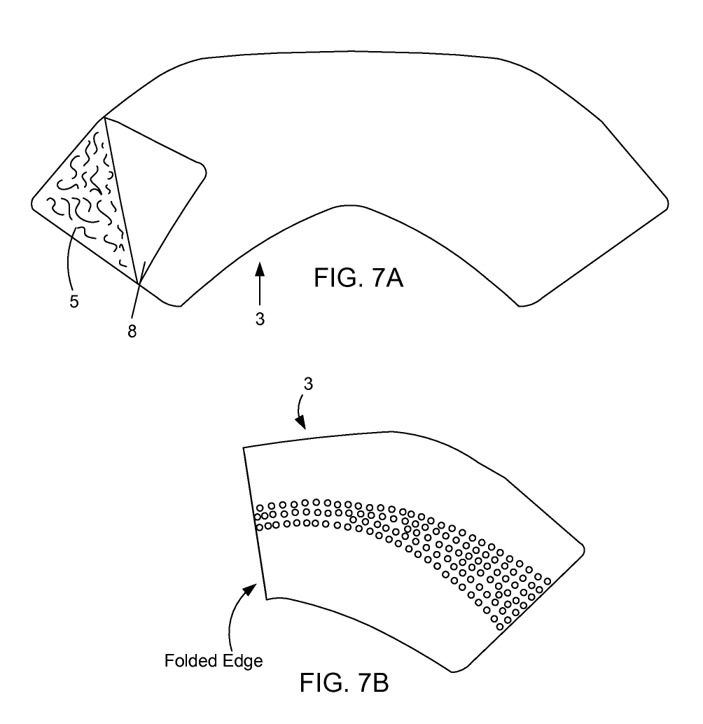

In FIG. 7A, the sexual partner has removed tooth cover 3 from its packaging. Sticky substance 5 is covered with wax paper 8. Prior to donning tooth cover 3 the sexual partner must first peal wax paper 8 from the bottom surface of tooth cover 3 to reveal sticky substance 5.

In a preferred embodiment, tooth cover 3 is packaged folded as shown in FIG. 7B. When unfolded, crease 10 will be exposed (FIG. 5), The sexual partner can use crease 10 to properly align tooth cover 3 with her teeth by ensuring that the crease is between either her two front top teeth or her two front bottom teeth.

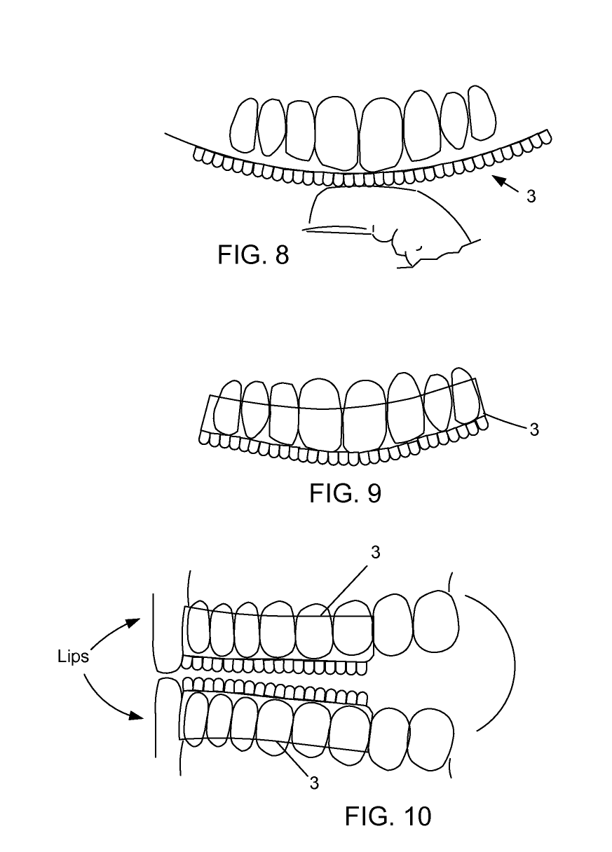

In FIG. 8 the sexual partner has begun to cover her top teeth with tooth cover 3. The sexual partner is pushing tooth cover 3 against her teeth with her thumb. Sticky substance 5 holds tooth cover 3 securely in place against her teeth.

In FIG. 9 the sexual partner has pushed the sides of tooth cover 3 against the sides of her teeth to finally secure placement. The ensure optimum hold, the sexual partner will then run her fingers along the sides and edges of her tooth cover 3 while press it against her teeth.

The steps described in reference to FIGS. 7-9 are repeated for the sexual partner's bottom teeth. Her top and bottom tooth then appear as shown in FIG. 10.

### Disposable Tooth Cover

In a preferred embodiment, for sanitary purposes each tooth cover 3 is a single-use disposable item. This adds to cleanliness of the tooth covers. In this preferred embodiment, the sexual partner purchases a package of tooth covers 3. The package may be purchased at a local drug store or market in packages of 12, 24 or 50, for example.

### Compactness

It should be recognized that tooth cover 3 is very compactable and can be easily packaged. For example, preferably tooth cover 3 is packaged in a folded configuration as shown in FIG. 7. Therefore many tooth covers 3 can be stored in a small box packager prior to purchase.

### Other Embodiments

In another preferred embodiment, sticky substance 5 is flavored. For example, a pleasant tasting mint flavor or chocolate flavor serves to mask the unpleasant taste of the male's erect penis when it is initially inserted into the sexual partner's mouth.

Also, in another preferred embodiment tooth cover 3 is fabricated from a clear polyurethane material. By being clear, the visual appearance of the sexual provider's mouth is more appealing in that tooth cover 3 becomes nearly invisible to the male.

Although the above-preferred embodiments have been described with specificity, persons skilled in this art will recognize that many changes to the specific embodiments disclosed above could be made without departing from the spirit of the invention. For example, nodules 4 were describe above as appearing as shown in FIG. 5. It should be recognize that nodules 4 can be modified in their appearance and number to form different patterns on tooth cover 3. Specifically, nodules 4 function to provide a textured surface to stimulate the males erect penis. Therefore, the exact configuration of the nodules can be according modified. Or, nodules 4 can be eliminated and the erect penis can contact directly the smooth top surface of tooth cover 3 for a less stimulating feeling. The less stimulating embodiment may allow ultimately allow the male to enjoy a longer sexual experience. Therefore, the attached claims and their legal equivalents should determine the scope of the invention.

What is claimed is:

1) A tooth cover for covering a sexual partner's teeth when a male's erect penis is inserted into the mouth of the sexual partner during oral sex, said disposable tooth cover comprising:
A. a flexible tooth cover body, comprising:
1. a bottom surface for attachment to the sexual partner's teeth, and
2. a top surface for providing a comfortable surface to receive said erect penis,
B. a sticky substance connected to said bottom surface for attaching said bottom surface to said sexual partner's teeth.

2) The tooth cover as in claim 1, wherein said tooth cover is a disposable tooth cover.

3) The tooth cover as in claim 2, wherein said tooth cover is a plurality of tooth covers and said plurality of tooth covers are sold together in packages.

4) The tooth cover as in claim 1 wherein said tooth cover body is fabricated of soft, flexible polyurethane.

5) The tooth cover as in claim 1 wherein said tooth cover is foldable for easy packaging.

6) The tooth cover as in claim 1, wherein said tooth cover is flat prior to attachment to the sexual partner's teeth.

7) The tooth cover as in claim 1, wherein said tooth cover comprises a crease for properly aligning said crease to the sexual partner's front two teeth.

8) The tooth cover as in claim 1, wherein said tooth cover comprises a plurality of nodules attached to the top of said top surface, wherein said plurality on nodules provide a textured contact surface to increase stimulation to the male's erect penis.

9) The tooth cover as in claim 1, wherein said tooth cover is two tooth covers, wherein the first of said two tooth covers is for covering the sexual partner's top teeth and wherein the second of said two tooth covers is for covering the sexual partner's bottom teeth.

10) The tooth cover as in claim 1, wherein said sticky substance is a denture adhesive.

11) The tooth cover as in claim 1, wherein said sticky substance is flavored to mask the unpleasant taste of the male's erect penis.

12) The tooth cover as in claim 1, wherein the top of said top surface is textured to increase stimulation to the male's erect penis.

13) A disposable tooth cover for covering a sexual partner's teeth when a male's erect penis is inserted into the mouth of the sexual partner during oral sex, said disposable tooth cover comprising:
A. a flexible tooth cover body, comprising:
1. a bottom surface for attachment to the sexual partner's teeth, and
2. a top surface for providing a comfortable surface to receive said erect penis,
B. a sticky substance connected to said bottom surface for attaching said bottom surface to said sexual partner's teeth,
C. a crease for properly aligning said crease to the sexual partner's front two teeth, and
D. a plurality of nodules attached to the top of said top surface, wherein said plurality on nodules provide a textured contact surface to increase stimulation to the male's erect penis,
wherein said tooth cover is foldable for easy packaging, wherein said tooth cover is flat prior to attachment to said sexual partner's teeth and wherein said sticky substance is flavored to mask the unpleasant taste of the male's erect penis.
